(19) United States
(12) Patent Application Publication
(10) Pub. No.: US 2025/0201426 A1
(43) Pub. Date: Jun. 19, 2025

(54) INFORMATION PROCESSING DEVICE, INFORMATION PROCESSING METHOD, AND PROGRAM

(71) Applicant: SONY GROUP CORPORATION, TOKYO (JP)

(72) Inventors: TAKUYA NAKAMURA, TOKYO (JP); HIROYASU BABA, TOKYO (JP); KANA MATSUURA, TOKYO (JP)

(21) Appl. No.: 18/849,688

(22) PCT Filed: Mar. 13, 2023
(86) PCT No.: PCT/JP2023/009516

§ 371 Date: Sep. 23, 2024

(30) Foreign Application Priority Data
Mar. 30, 2022 (JP) 2022-055245

(51) Int. Cl. G16H 50/70

(52) U.S. Cl. CPC G16H 50/70

(57) ABSTRACT

The present technology relates to an information processing device, an information processing method, and a program enabling to improve efficiency of surgery-related work. An information processing device includes: an analysis unit configured to analyze data regarding surgery; a search condition acquisition unit configured to acquire a search condition for searching for a reference case to be compared with an evaluation case as an evaluation target; a search unit configured to search for the reference case matching the search condition from a database in which results of analysis by the analysis unit are accumulated; a calculation means configured to calculate an evaluation point of the evaluation case by using the evaluation case and the reference case obtained by searching with the search unit; a presentation means configured to present the evaluation point calculated by the calculation means to a user; a feedback acquisition means configured to acquire feedback given by the user on the evaluation point presented by the presentation means; and an update unit configured to update the evaluation point obtained by the calculation means, in a case where feedback is acquired by the feedback acquisition means. The present technology can be applied to, for example, an information processing device that evaluates a case related to surgery.

### TECHNICAL FIELD

The present technology relates to an information processing device, an information processing method, and a program, and for example, relates to an information processing device, an information processing method, and a program capable of providing a means for improving efficiency of surgery-related work.

### BACKGROUND ART

In recent years, with diversification and complication of surgery, costs related to surgery have dramatically increased in hospitals. On the other hand, there is a fact that hospitals obtain more than half of profits by surgery. Under such circumstances, hospitals have paid attention not only to the number of surgeries but also to cost reduction by improving quality and efficiency of the surgery.

As a related art intended to improve efficiency of surgery, for example, Patent Document 1 discloses a technique of providing a camera in a surgery-related facility, calculating a surgery execution time from a movement mode of a doctor, a medical staff member, equipment, and the like detected using the camera, calculating a balance in surgery from the calculated surgery execution time and a time unit cost of a labor expense and the like, and displaying the calculated balance and the movement mode.

### CITATION LIST

### Patent Document

Patent Document 1: Japanese Patent Application Laid-Open No. 2014-209350

### SUMMARY OF THE INVENTION

### Problems to be Solved by the Invention

However, in Patent Document 1, it is possible to determine a delay or the like for individual work, but it is difficult to specify which work is delayed throughout, so that there has been a possibility that sufficiently improved efficiency of surgery-related work cannot be achieved. It is desired to provide a means for further improving efficiency of surgery-related work.

The present technology has been made in view of such a situation, and an object of the present technology is to provide a means for improving efficiency of surgery-related work.

### Solutions to Problems

An information processing device according to one aspect of the present technology is an information processing device including: an analysis unit configured to analyze data regarding surgery; a search condition acquisition unit configured to acquire a search condition for searching for a reference case to be compared with an evaluation case as an evaluation target; a search unit configured to search for the reference case matching the search condition from a database in which results of analysis by the analysis unit are accumulated; a calculation means configured to calculate an evaluation point of the evaluation case by using the evaluation case and the reference case, the reference case being obtained by searching with the search unit; a presentation means configured to present the evaluation point calculated by the calculation means to a user; a feedback acquisition means configured to acquire feedback given by the user on the evaluation point presented by the presentation means; and an update unit configured to update the evaluation point obtained by the calculation means, in a case where feedback is acquired by the feedback acquisition means.

An information processing method according to one aspect of the present technology is an information processing method including, by an information processing device: analyzing data regarding surgery; acquiring a search condition for searching for a reference case to be compared with an evaluation case as an evaluation target; searching for the reference case matching the search condition from a database in which results of analysis are accumulated; calculating an evaluation point of the evaluation case by using the evaluation case and the reference case, the reference case being obtained by the searching; presenting the calculated evaluation point to a user; acquiring feedback given by the user on the presented evaluation point; and updating the evaluation point in a case where the feedback is acquired.

A program according to one aspect of the present technology is a program for causing a computer to execute processing including a step of: analyzing data regarding surgery; acquiring a search condition for searching for a reference case to be compared with an evaluation case as an evaluation target; searching for the reference case matching the search condition from a database in which results of analysis are accumulated; calculating an evaluation point of the evaluation case by using the evaluation case and the reference case, the reference case being obtained by the searching; presenting the calculated evaluation point to a user; acquiring feedback given by the user on the presented evaluation point; and updating the evaluation point in a case where the feedback is acquired.

In the information processing device, the information processing method, and the program of one aspect of the present technology, data regarding surgery is analyzed, a search condition for searching for a reference case to be compared with an evaluation case as an evaluation target is acquired, a reference case matching the search condition is searched for from a database in which results of analysis by the analysis unit are accumulated, an evaluation point of the evaluation case is calculated by using the evaluation case and the reference case, the reference case being obtained by searching, the calculated evaluation point is presented to a user, feedback given by the user on the presented evaluation point is obtained, and the evaluation point is updated in a case where the feedback is acquired.

Note that the information processing device may be an independent device or an internal block constituting one device.

Note that a program to be provided may be transmitted via a transmission medium or be recorded on a recording medium.

### BRIEF DESCRIPTION OF DRAWINGS

FIG. 1 is a diagram for explaining an outline of a surgery-related work efficiency improvement flow to which the present technology is applied.

FIG. 2 is a diagram for explaining a configuration example of an information processing system.

FIG. 3 is a diagram for explaining data recorded in a database.

FIG. 4 is a diagram for explaining a function of a data processing unit.

FIG. 5 is a flowchart for explaining processing related to evaluation.

FIG. 6 is a diagram illustrating an example of a search condition input screen.

FIG. 7 is a view for explaining a problem presentation screen.

FIG. 8 is a diagram for explaining a recording medium.

### MODE FOR CARRYING OUT THE INVENTION

Hereinafter, a mode for carrying out the present technology (hereinafter referred to as an embodiment) will be described.

Since the present technology can be applied to an information processing system for improving efficiency of surgery-related work, a description will be given with improvement of efficiency of surgery-related work as an example. As viewpoints to be focused on for improving efficiency of surgery-related work, there are a viewpoint on an operation side, a viewpoint on a doctor and worker (hereinafter, referred to as staff member) side such as a medical staff member, a viewpoint from a case side, and the like. The viewpoint on the operation side is an important viewpoint for grasping an operation state of resources such as staff members and equipment. The viewpoint on the staff member side is an important viewpoint in recognition of a scene and support of workflow in surgery-related work including surgery preparation and the like. The viewpoint from the case side is an important viewpoint in presenting information for improving quality of treatment to a doctor (hereinafter, referred to as a surgeon) and a staff member who perform surgery-related work.

Conventionally, it has not been possible to visualize information based on these viewpoints in an easy-to-understand manner. Therefore, finding, specification, and the like of problems in surgery-related work are difficult, which has been an obstacle in achieving sufficiently improved efficiency of the surgery-related work. Therefore, in the following embodiment, it is possible to further improve efficiency of surgery-related work by visualizing information based on the viewpoints described above in a more easy-to-understand manner.

Specifically, by visualizing an operation status of a staff member, equipment, and the like from various viewpoints, it is possible to facilitate analysis for finding, specifying, and the like of problems in surgery-related work, thereby supporting creation of efficient workflow that enables cost reduction and time reduction in the surgery-related work. Furthermore, by enabling optimal resource disposition based on the visualized information, it is possible to support further efficiency improvement of the workflow.

### <Outline of Surgery-Related Work Efficiency Improvement Flow>

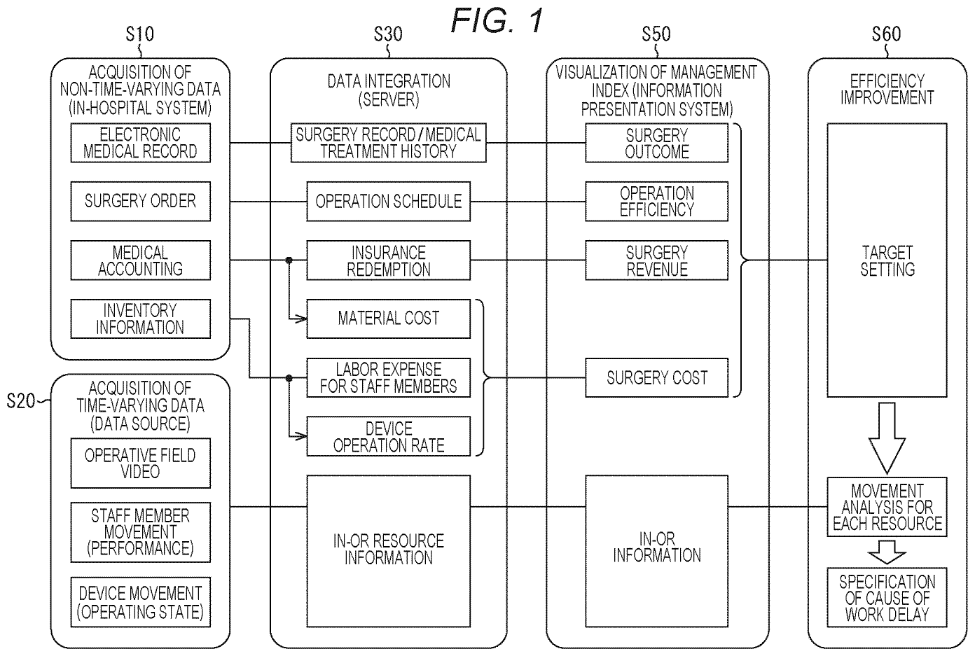

FIG. 1 is a schematic diagram for explaining a surgery-related work efficiency improvement flow according to an embodiment of the present disclosure. As illustrated in FIG. 1, in the surgery-related work efficiency improvement flow according to the present embodiment, the following steps are executed: step S10 of acquiring data that does not change with time (hereinafter, referred to as non-time-varying data); step S20 of acquiring data that changes with time (hereinafter, referred to as time-varying data); step S30 of integrating these pieces of data; step S50 of presenting information (hereinafter, referred to as surgery-related information) based on the integrated data to a user; and step S60 in which the user improves efficiency of workflow of surgery-related work, disposition of resources, and the like on the basis of the presented surgery-related information.

The non-time-varying data acquired in step S10 is, for example, data registered by a doctor, a scopist, an anesthesiologist, a nurse, a staff member such as an accounting clerical worker, or the like in a system (hereinafter, referred to as an in-hospital system) in a hospital. The non-time-varying data can include, for example, static data regarding an electronic medical record, a surgery order, medical accounting, inventory information, and the like.

The “electronic medical record” may be a medical record in which information regarding medical treatment performed on a patient as a surgery target is collected and digitized.

The “surgery order” is, for example, information regarding a schedule or the like of every surgery-related work, and can include information regarding a surgical procedure, an engaged medical staff member and medical team (hereinafter, simply referred to as a staff member), used equipment, and the like, for example, in addition to a place of surgery (hereinafter, also referred to as an operating room (OR)), a date and time.

The “medical accounting” is, for example, information regarding a balance of payments of every surgery-related work, and can include, for example, information regarding an expense required for the surgery-related work, a billing amount for a patient, an insurance redemption amount, and the like. Note that the expense required for the surgery-related work may include a labor expense of a doctor, a staff member, and the like involved in surgery, a cost of chemical agents, consumables, and the like required for surgery, a usage charge of an operating room and equipment, a depreciation expense, and the like.

The “inventory information” may be, for example, information regarding inventory and order of devices, supplies, chemical agents, consumables, and the like (hereinafter, also referred to as equipment). Note that the chemical agents can include, for example, an anesthetic, a blood preparation, and the like used for surgery. Furthermore, consumables can include gauze, surgical gloves, and the like. The time-varying data acquired in step S20 is, for example, data acquired from a camera attached to a surgery-related facility such as an operating room, a sensor attached to a staff member or a device, or the like (hereinafter, referred to as a data source). The time-varying data can include, for example, an operative field video, a staff member movement (hereinafter, also referred to as performance), device movement (hereinafter, also referred to as an operating state), and the like.

The “operative field video image” may be, for example, video data acquired by a camera disposed in an operating room or a peripheral/related facility thereof.

The “staff member movement (performance)” may be information regarding, for example, a position of a staff member or a device specified by analyzing a video acquired by the camera, and a movement trajectory of a position specified on the basis of a signal from a global positioning system (GPS) oscillator or the like attached to a staff member or a device.

The “device movement (operating state)” may be, for example, information regarding an operating state of a device used in surgery-related work, such as an electrocardiogram monitor or an electric scalpel.

In step S30, the non-time-varying data acquired in step S10 and the time-varying data acquired in step S20 are collected and accumulated in a server disposed inside or outside the hospital. Furthermore, in step S30, from the collected data, a surgery record/medical treatment history, an operation schedule, insurance redemption, a material cost, a labor expense for staff members, a device operation rate, in-OR resource information, and the like may be derived.

The “surgery record/medical treatment history” may be, for example, a data group in which records of surgery-related work performed in the hospital and medical treatment histories specified from the electronic medical records or the like are accumulated.

The “operation schedule” may be, for example, a data group related to a schedule of a surgery-related facility, device, a staff member, and the like planned on the basis of the surgery order or the like.

The “insurance redemption” may be, for example, a data group regarding an insurance redemption amount of every surgery-related work or medical treatment specified on the basis of the medical accounting or the like.

The “material cost” may be, for example, a data group related to a labor expense for each staff member, a running cost caused by using a device, a unit price of consumables, and the like specified on the basis of the surgery order or the like.

The “device operation rate” may be, for example, a data group related to an operation rate of a device used in surgery-related work, which is specified on the basis of the surgery order or the like.

The “in-OR resource information” may be, for example, a data group in which information regarding resources such as staff members and devices specified from the time-varying data is collected.

In step S50, for example, a surgery outcome, operation efficiency, surgery revenue, a surgery cost, in-OR information, and the like are visualized and presented to the user, on the basis of the data group integrated in the server in step S30.

The “surgery outcome” may be, for example, information in which a result of evaluating work or a result in surgery-related work of every surgeon or staff member is visualized.

The “operation efficiency” may be, for example, information in which a degree of efficiency improvement of operation histories of resources, such as surgeons, staff members, and equipment, is visualized.

The “surgery revenue” may be, for example, information in which a balance of payments in surgery-related work is visualized.

The “surgery cost” may be, for example, information in which a running cost such as a labor expense and a material cost required for surgery-related work is visualized.

The “in-OR information” may be, for example, information in which “in-OR resource information” is visualized, information in which a result of analyzing “in-OR resource information” is visualized, or the like.

In step S60, for example, the user sets an item to be targeted for efficiency improvement on the basis of the information such as the surgery outcome, the operation efficiency, the surgery revenue, and the surgery cost visualized in step S50. Then, the user analyzes a movement of every resource in the targeted item on the basis of the visualized in-OR information, and specifies a cause of a work delay, to improve the efficiency of surgery-related work.

### <Configuration Example of Information Processing System>

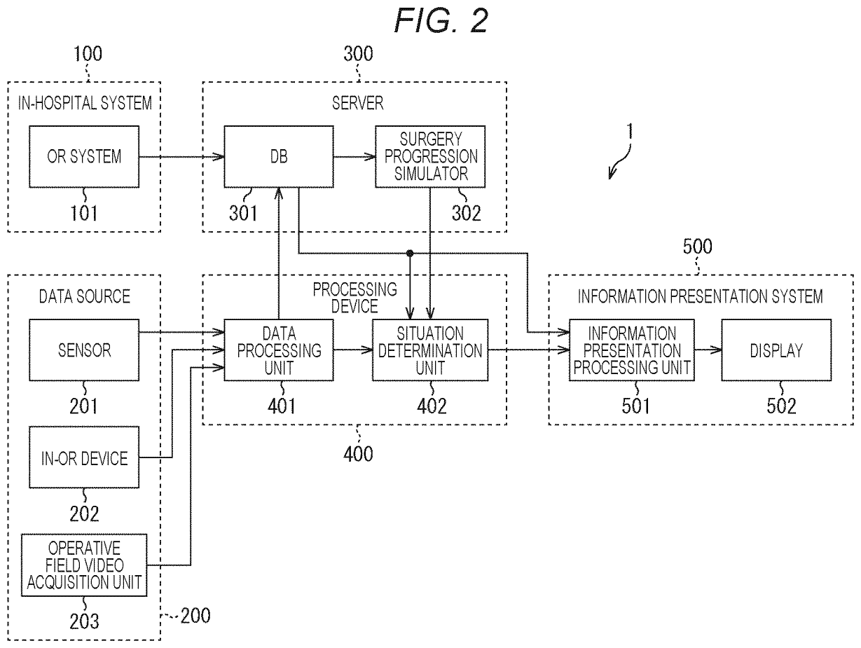

FIG. 2 is a block diagram illustrating a schematic configuration example of the information processing system according to the present embodiment. As illustrated in FIG. 2, an information processing system 1 according to the present embodiment includes an in-hospital system 100, a data source 200, a server 300, a processing device 400, and an information presentation system 500.

The data source 200, the server 300, the processing device 400, and the information presentation system 500 may be communicably connected to each other via a predetermined network such as, for example, a local area network (LAN), a wide area network (WAN), the Internet, or a mobile communication network.

The in-hospital system 100 includes, for example, an operating room system (hereinafter, referred to as an OR system) 101 installed in a medical facility such as a hospital or a clinic, and the in-hospital system 100 may be a system that executes registration and management of non-time-varying data such as electronic medical records, surgery orders, medical accounting, and inventory information. Note that the medical facility is not necessarily a facility installed in one site, and may be a facility disposed in a plurality of sites in a distributed manner.

The data source 200 includes, for example, a sensor 201 such as a GPS transmitter or a radio frequency identifier (RFID) tag, an in-OR device 202 such as an electrocardiogram monitor or an electric scalpel, and an operative field video acquisition unit 203 such as a camera, and acquires time-varying data such as an operative field video, a staff member movement, and a device movement. Note that the sensor 201, the in-OR device 202, and the operative field video acquisition unit 203 may be disposed in a medical facility in which the in-hospital system 100 is installed, or on a doctor, a staff member, or the like working in the medical facility.

The server 300 includes, for example, a database 301 and a surgery progression simulator 302. In the database 301, for example, non-time-varying data registered in the in-hospital system 100 and time-varying data acquired by the data source 200 are collected and accumulated. For example, by using the data accumulated in the database 301, the surgery progression simulator 302 performs reproduction, analysis, simulation, and the like of a flow of the entire surgery (entire process), each process in the entire surgery, work contents of each work section in each process, a tool used in each work section, a movement of a device and a staff member in each work section, and the like.

Note that, in the present description, the “process (not including the entire process)” may be a section (also referred to as a first section) obtained by sectioning the entire process of the surgery for every process according to the work content, and the work section may be a section (also referred to as a second section) in which each process is further divided for every work content.

The processing device 400 includes, for example, a data processing unit 401 and a situation determination unit 402. The data processing unit 401 converts, for example, the time-varying data acquired by the data source 200 into a necessary format. The time-varying data subjected to the format conversion is transmitted to the database 301 and accumulated, for example, and is also input to the situation determination unit 402 as necessary. The situation determination unit 402 determines a situation at a certain time point in surgery that is currently in progress or surgery performed in the past, for example, on the basis of contents and results of reproduction, analysis, simulation, and the like input from the surgery progression simulator 302, time-varying data input from the data processing unit 401, and the like.

The information presentation system 500 includes, for example, an information presentation processing unit 501 and a display 502. The information presentation processing unit 501 generates data for displaying and reproducing, in a mode easily recognizable by a user, a situation determination result and contents and results of reproducing, analyzing, and simulating a flow of the entire surgery, contents of work in each work section, a tool used in each work section, a movement of a device and a staff member in each work section, and the like (surgery-related information), which are input from the situation determination unit 402. The display 502 presents the above-described contents, the results, the situation determination result, and the like to the user by displaying and reproducing the data generated by the information presentation processing unit 501.

Note that the “mode that is easily recognizable by a user” in the present description may be, for example, an image, sound, vibration, or the like. Furthermore, “visualization” in the present description may include not only enabling visual recognition but also enabling auditory or tactile recognition.

### <Example of Resource Information Accumulated in Database>

Here, an example of resource information accumulated in the database 301 of the server 300 (referred to as resource information) will be described. In the present description, the resource may be a resource such as a person, an object, or a facility engaged in surgery-related work.

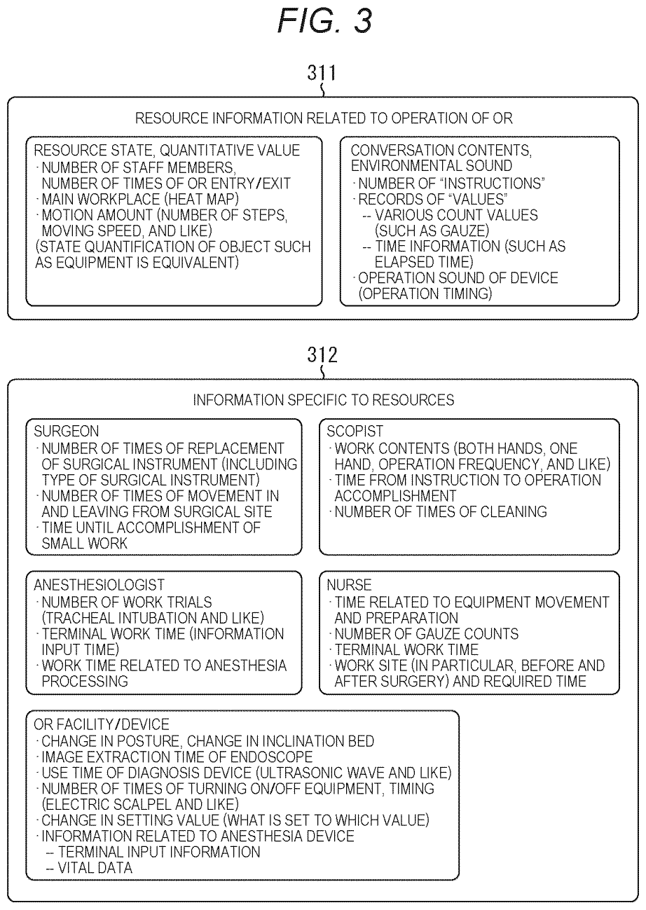

FIG. 3 is a schematic diagram illustrating an example of resource information accumulated in the database according to the present embodiment. As illustrated in FIG. 3, for example, “resource state, quantitative value” and “conversation contents, environmental sound” may be accumulated in the database 301 as resource information 311 related to operation of the OR.

For example, in a case where the resource is a person such as a surgeon or a staff member, the “resource state, quantitative value” can include the number of staff members, the number of times of OR entry/exit of each staff member, a main workplace (heat map) of each staff member, and a motion amount (number of steps, moving speed, and the like) of each staff member. Note that, also in a case where the resource is an object such as a device, values can be accumulated by similar quantification.

The “conversation contents, environmental sound” can include, for example, the number of instructions to a staff member, records of various “values”, operation sound of the device, and the like. Note that the records of “values” can include records of a count value of supplies such as gauze, records of time information such as an elapsed time from a start of surgery, and the like. Furthermore, the operation sound of the device can be used to specify an operation timing of each device.

Furthermore, in the database 301, for example, information regarding a surgeon, a scopist, an anesthesiologist, a nurse, an OR facility/device, and the like may be accumulated as information 312 specific to resources.

Information specific to the “surgeon” can include, for example, the number of times of replacement of a surgical instrument (which can include a type of the surgical instrument), the number of times of movement in and leaving from a surgical site, a time until accomplishment of small work, and the like.

Information specific to the “scopist” can include, for example, information such as work contents (both hands, one hand, operation frequency, and the like), a time from an instruction to operation accomplishment, and the number of times of cleaning.

Information specific to the “anesthesiologist” can include, for example, information such as the number of work trials (tracheal intubation and the like), a terminal work time (information input time), and a work time related to anesthesia processing.

Information specific to the “nurse” can include, for example, information such as a time related to equipment movement and preparation, the number of gauze counts, a terminal work time, and a work site (in particular, before and after surgery) and a required time.

Information specific to the “OR facility/device” can include, for example, information such as a change in posture, a change in inclination of a bed, an image extraction time of an endoscope, a use time of a diagnosis device (ultrasonic wave and the like), the number of times of turning on/off equipment, a timing (electric scalpel and the like), a change in a setting value (what is set to which value, and the like), and information related to an anesthesia device (terminal input information, vital data).

### <Functional Configuration Example of Processing Device>

The processing performed in the information processing system 1 illustrated in FIG. 2 includes processing of presenting a problem point to the user, receiving feedback from the user on the presented problem point, and performing update on the basis of the feedback. The processing related to series of evaluation will be described.

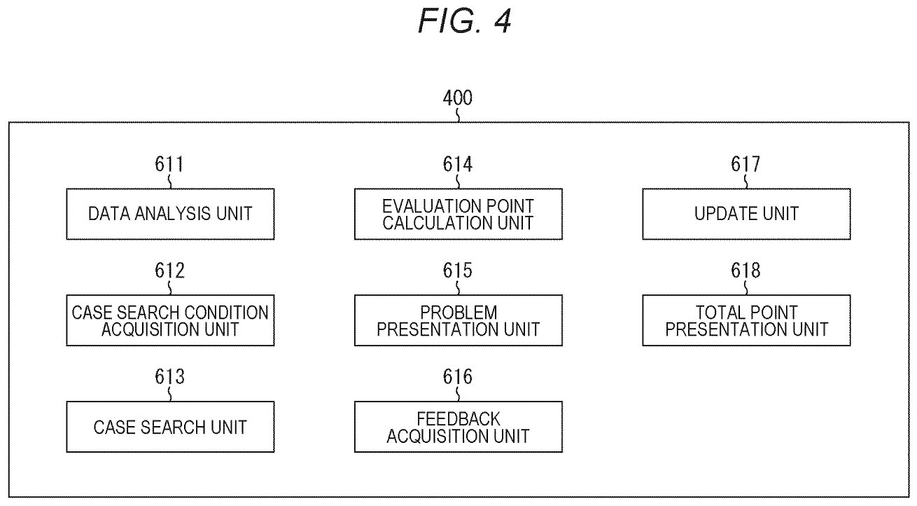

FIG. 4 is a functional block diagram of the processing device 400 related to the processing related to the evaluation. A data analysis unit 611 generates data to be stored in the database 301 described with reference to FIG. 3, for example, by using the data obtained from the data source 200. For example, in a case where a video obtained by capturing an image of the inside of the OR system 101 is acquired as a data source, by detecting a person or an object (device) and performing three-dimensional tracking to estimate a posture or a position of the person, a position of the person is detected, such as, for example, whether the person is around the bed or at a distant position, or work (task) performed by the person is estimated together with operation information of the device or the like. Results of the analysis by the data analysis unit 611 are accumulated in the database 301.

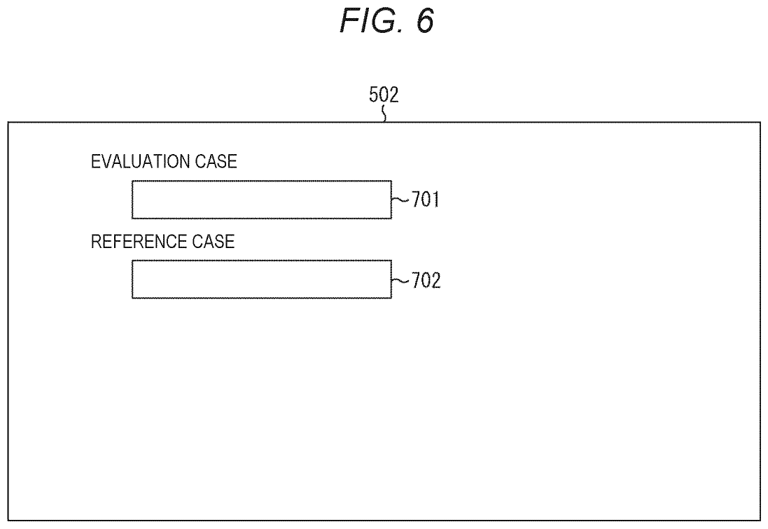

A case search condition acquisition unit 612 acquires a search condition input by the user when searching for a case to be an evaluation target and a case to be compared with the evaluation target. For example, the search condition input on a screen to be described later with reference to FIG. 6 is acquired.

A case search unit 613 searches and reads, from the database 301, an evaluation case matching the search condition acquired by the case search condition acquisition unit 612. The evaluation case obtained by the searching with the case search unit 613 is supplied to an evaluation point calculation unit 614. The evaluation point calculation unit 614 calculates an evaluation point of the evaluation case set as the evaluation target, by using a reference case.

A problem point presentation unit 615 presents a problem point in the evaluation target case to the user. A feedback acquisition unit 616 receives feedback from the user on the problem point presented by the problem point presentation unit 615, and acquires the feedback. When the feedback is acquired, an update unit 617 updates the evaluation point.

A total point presentation unit 618 presents evaluation including the feedback result to the user in a format of a total point set as an evaluation result of the evaluation target case.

### <Flow Regarding Evaluation>

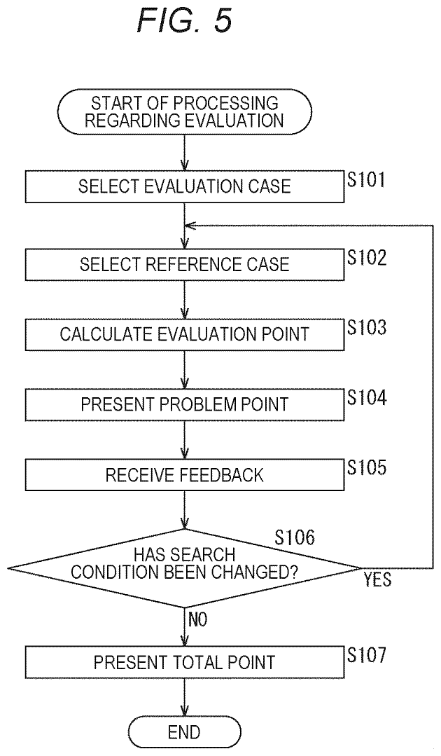

FIG. 5 is a flowchart for explaining processing related to presentation of evaluation for a predetermined case performed by the processing device 400 illustrated in FIG. 4. The description will be continued on the assumption that the processing of the flowchart illustrated in FIG. 5 is a result of analysis performed by the data analysis unit 611 and is started in a case where a plurality of reference cases is stored in the database 301.

In step S101, an evaluation case set as the evaluation target is selected. In step S102, a reference case to be compared with the evaluation case is selected. FIG. 6 is a view illustrating an example of a screen displayed on the display 502 when the evaluation case or the reference case is selected.

The screen displayed on the display 502 is, for example, a screen for setting each of the evaluation case to be the evaluation target and the reference case to be referred to in comparing and considering the evaluation case. In the screen example illustrated in FIG. 6, an evaluation case input field 701 for input of a search condition (information) related to the evaluation case and a reference case input field 702 for inputting a search condition (information) related to the reference case are provided.

In the evaluation case input field 701, information regarding a case for which the user wants to know evaluation, for example, a surgeon, a surgery name, a surgery date, or the like is input as a keyword.

For example, in a case where login using a user ID or a password is performed before the screen illustrated in FIG. 6 is displayed, the most recent surgery related to the user identified by the login may be input in advance in the evaluation case input field 701. The evaluation case input field 701 may be in a format of a pull-down menu, and a list of surgeries related to the user identified by login may be displayed and selected.

In the reference case input field 702, information regarding a case as a comparison target for evaluating the evaluation case is input. Examples thereof include an operating surgeon, a surgery name, and a period of a case set as a search target. Basically, a case that is the same as or similar to the evaluation case is to be the reference case. A keyword associated with the evaluation case specified by the information input in the evaluation case input field 701 may be input in advance in the reference case input field 702 (displayed before input by the user).

The reference case is a case of the same type as the evaluation case stored in the database 301 (FIG. 2) or a case in which the same surgical procedure is adopted, and statistics and an average value of these cases are used for calculation of an evaluation point described later.

The case search condition acquisition unit 612 presents the screen as illustrated in FIG. 6 to the user, acquires a condition of the evaluation case to be an evaluation target input by the user, and acquires a condition of the reference case to be compared with the evaluation case input by the user. The case search unit 613 searches and reads, from the database 301, a case matching the condition for the evaluation case acquired by the case search condition acquisition unit 612, and searches and reads, from the database 301, a case matching the condition for the reference case.

Regarding the reference case, a plurality of cases matching the search condition input in the reference case input field 702 is obtained by searching (also including a case where only one search result is found).

In step S103, an evaluation point is calculated by the evaluation point calculation unit 614. The evaluation point is calculated for each item on the basis of the evaluation case and the reference case obtained by searching. An example of an item and a method for calculating the evaluation point for the item will be described below.

$$Water balance ( out ) = balance amount / body weight / reference value \qquad ( 1 )$$

Formula (1) is a formula for calculating an evaluation point regarding an item of water balance such as a blood loss and a urine amount. Formula (1) is a formula of dividing, by a reference value, a value obtained by dividing a balance amount by a body weight of a patient with an evaluation symptom. The reference value is an average value of water balance calculated on the basis of Formula (1) in a reference case obtained by searching. Evaluation is better as the value calculated in Formula (1) is lower.

$$Cost = actual expense / reference expense \qquad ( 2 )$$

Formula (2) is a formula for calculating an evaluation point regarding an item of a cost required for the evaluation case. Formula (2) is a formula of dividing, by a reference expense, an actual expense such as an expense actually required for surgery for an evaluation symptom, for example, a labor expense for people involved in surgery and a purchase expense of chemical agents. The reference expense is an average value of costs calculated on the basis of Formula (2) in a reference case obtained by searching. Evaluation is better as the value calculated in Formula (2) is lower.

$$Preoperative time = elapsed time before surgery / preoperative reference time \qquad ( 3 )$$

Formula (3) is a formula for calculating an evaluation point regarding an item of a preoperative time required before surgery. Formula (3) is a formula of dividing, by a reference time, a time taken for preparation for an evaluation symptom before surgery. The reference time is an average value of an elapsed time before surgery calculated on the basis of Formula (3) in a reference case obtained by searching. Evaluation is better as the value calculated in Formula (3) is lower.

$$Intraoperative time = elapsed time during surgery / reference time during surgery \qquad ( 4 )$$

Formula (4) is a formula for calculating an evaluation point regarding an item of an intraoperative time required during surgery. Formula (4) is a formula of dividing, by a reference time, a time taken for surgery for an evaluation symptom before surgery. The reference time is an average value of an elapsed time during surgery calculated on the basis of Formula (4) in a reference case obtained by searching. Evaluation is better as the value calculated in Formula (4) is lower.

$$Postoperative time = elapsed time after surgery / reference time after surgery \qquad ( 5 )$$

Formula (5) is a formula for calculating an evaluation point regarding an item of a postoperative time required after surgery. Formula (5) is a formula of dividing, by a reference time, a time taken for post-treatment of an evaluation symptom after surgery. The reference time is an average value of an elapsed time after surgery calculated on the basis of Formula (5) in a reference case obtained by searching. Evaluation is better as the value calculated in Formula (5) is lower.

$$Prognosis = number of days for discharge \\ after surgery / reference number of days for discharge \qquad ( 6 )$$

Formula (6) is a formula for calculating an evaluation point regarding an item of prognosis, with a time required after surgery until discharge as prognosis. Formula (6) is a formula of dividing a time taken from surgery to discharge from the hospital by a reference time. The reference time is an average value of prognosis calculated based on Formula (6) in a reference case obtained by searching. Evaluation is better as the value calculated in Formula (6) is lower.

Note that, since the calculation formula of the item of prognosis includes the number of days for discharge, for example, in a case where an evaluation point is calculated immediately after surgery (during a period before discharge), the evaluation point cannot be calculated on the basis of Formula (6). Therefore, in such a case, for example, the evaluation point regarding prognosis is set to 1.

The evaluation points calculated on the basis of Formulas (1) to (6) are evaluation points relating to the patient. As such an evaluation point classified into a patient background, the evaluation point may be calculated in consideration of a body mass index (BMI), a blood pressure, a past history, and the like of the patient.

For example, in a case where the BMI is considered as a patient background, +5 may be applied as the evaluation point in a case of a patient with a BMI of 40, +5 may be applied as the evaluation point in a case of a patient with a blood pressure of 150 or more, and +10 may be applied as the evaluation point in a case of a patient with a past history. The evaluation point varies depending on a value of the BMI, a value of the blood pressure, and contents of the past history.

Items related to a staff member (medical worker, and the like) include a stability degree, a busy degree, and total workload.

$$Stability degree in surroundings of bed ( coefficient of variation ) = standard deviation of movement amount in surroundings \\ of bed / average value / reference coefficient of variation \qquad ( 7 )$$

For example, it is estimated that a staff member is in a busy state in a case where a movement amount of the staff member during surgery is large, and it is estimated that the staff member is not in a busy state, in other words, the staff member is stable, in a case where the movement amount is small. Formula (7) is a formula for calculating an evaluation point regarding an item of a stability degree representing busyness during surgery.

Formula (7) is a formula in which a standard deviation of a movement amount of a staff member in surroundings of a surgical bed is divided by an average value, and the value obtained by the division is divided by a reference coefficient of variation. The average value is an average value of a standard deviation of a movement amount in surroundings of a bed calculated in a reference case obtained by searching. The reference coefficient of variation is an average value of a stability degree (coefficient of variation) calculated on the basis of Formula (7) in a reference case obtained by searching. Evaluation is better as the value calculated in Formula (7) is lower.

$$Stability degree outside of \\ surroundings of bed ( coefficient of variation ) = standard deviation of movement amount outside of surroundings \\ of bed / average value / reference coefficient of variation \qquad ( 8 )$$

Formula (8) is a formula for calculating an evaluation point regarding an item of a stability degree representing busyness during surgery. Formula (8) is a formula in which a standard deviation of a movement amount of a staff member at a position separated by a predetermined distance from surroundings of a surgical bed is divided by an average value, and the value obtained by the division is divided by a reference coefficient of variation.

The average value is an average value of a standard deviation of a movement amount outside of surroundings of a bed calculated in a reference case obtained by searching. The reference coefficient of variation is an average value of a stability degree (coefficient of variation) calculated on the basis of Formula (8) in a reference case obtained by searching. Evaluation is better as the value calculated in Formula (8) is lower.

The evaluation point of the stability degree calculated by Formula (7) or Formula (8) is an index with which a degree of variation can be compared regardless of a case and a procedure, and an index closer to 1 indicates that the variation is lower and indicates a higher stability degree.

$$Busy degree ( standard deviation ) = standard deviation \\ of movement amount / reference standard deviation value \qquad ( 9 )$$

Formula (9) is a formula for calculating an evaluation point regarding an item of a busy degree representing busyness. Formula (9) is a formula of dividing a standard deviation of a movement amount by a reference standard deviation value. The reference standard deviation is an average value of a busy degree (standard deviation) calculated on the basis of Formula (9) in a reference case obtained by searching. Evaluation is better as the value calculated in Formula (9) is lower. The busy degree calculated in Formula (9) is an index for simply comparing magnitude of variation, and a value of 1 or more indicates being busy.

$$Total workload = ( total movement amount / reference case total movement amount ) \times ( average number of people / reference average number of people ) \qquad ( 10 )$$

Formula (10) is a formula for calculating an evaluation point regarding an item of total workload. Formula (10) is a formula of dividing a total movement amount by a reference case total movement amount, and multiplying the resulting value by a value obtained by dividing an average number of people by a reference average number of people.

The reference case total movement amount is a value (average value) obtained by dividing, by the number of cases, a value obtained by cumulatively adding a movement amount of a reference case obtained by searching. The average number of people is an average number of people of a total number of staff members involved in an evaluation case. The reference average number of people is a value obtained by dividing a total number of staff members of a reference case obtained by searching by the number of reference cases obtained by searching. Evaluation is better as the value calculated in Formula (10) is lower. The total workload calculated in Formula (10) is an index indicating that the workload is too much as the total workload is larger than 1.

Items related to manipulation include a task processing time achievement degree, a task order matching degree, and a patient care degree.

$$Task processing time achievement degree = ( each task / time sum of reference times ) / total number of tasks \qquad ( 11 )$$

Formula (11) is a formula for calculating an evaluation point regarding an item of a task processing time achievement degree, which is an index indicating whether or not a time taken to process one task is appropriate. Formula (11) is a formula of dividing, by the total number of tasks, a value obtained by dividing each task time by the sum of the reference times. The reference time is an average value of the sum of times related to each task in a reference case obtained by searching. Evaluation is better as the value calculated in Formula (11) is lower.

$$Task order matching degree = number of order - matching blocks / number of blocks in order task \qquad ( 12 )$$

Formula (12) is a formula for calculating an evaluation point regarding an item of a task order matching degree, which is an index indicating whether or not the tasks are performed in accordance with an order of the tasks. Formula (12) is a formula of dividing the number of blocks in which an order of task processing matches, by the number of blocks of the order task.

In a case where the blocks of the tasks having a determined order are in the order of 1→2→3, for example, when the tasks are processed in the order of 1→3→2→3, the processing is performed in an order different from the predetermined order of the processing of the tasks. In such a case, the tasks are not processed in accordance with the predetermined order of processing of the tasks, and the task order matching degree obtained by Formula (12) is calculated as an index of a deviation degree thereof. Evaluation is better as the task order matching degree is higher.

$$Patient care degree = ( number of times of patient care task \times time ) / \\ ( reference number of times of care \times time ) \qquad ( 13 )$$

Formula (13) is a formula for calculating an evaluation point regarding an item of a patient care degree, which is an index of how much the patient has been cared for. Formula (13) is a formula of dividing a value obtained by multiplying the number of times of performing the patient care task by a time, by a value obtained by multiplying a reference number of times of care by a time. The reference number of times of care is an average value of the number of times of patient care task for a reference case obtained by searching. Evaluation is better as the value calculated in Formula (13) is higher.

Formulas (1) to (13) are examples, and the evaluation points of individual items described above can be calculated by different calculation formulas. Here, the description will be continued with an example in which the evaluation points are calculated by Formulas (1) to (13). However, a case where the evaluation points are calculated by other arithmetic formulas, a case where the evaluation points calculated by other arithmetic formulas are further added to Formulas (1) to (13), and the like are also the application range of the present technology.

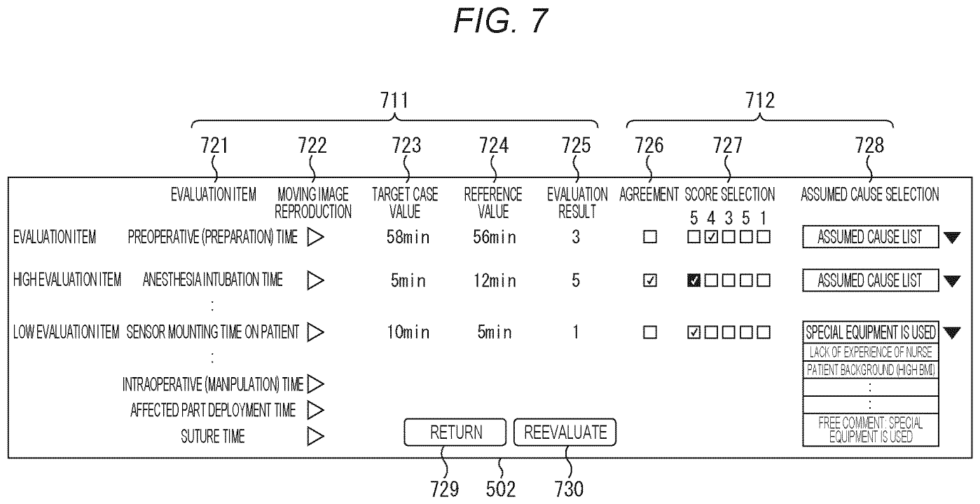

Returning to the description of the flowchart of FIG. 5, when the evaluation point is calculated in step S103, a problem point is presented to the user by the problem point presentation unit 615 in step S104. FIG. 7 is a view illustrating an example of a screen including a problem point to be presented to the user in step S104.

The screen illustrated in FIG. 7 is provided with a problem presentation part 711 that presents a problem and a feedback input part 712 that acquires feedback from the user.

The problem presentation part 711 includes an evaluation item display part 721 that displays an evaluation item, a moving image reproduction button 722 operated when a video is reproduced, a target case value display part 723 that displays a value of a case as an evaluation target, a reference value display part 724 that displays a reference value, and an evaluation result display part 725 that displays an evaluation point.

The feedback input part 712 includes, an agreement confirmation check part 726 that is checked in a case of agreeing on evaluation displayed in the evaluation result display part 725, a score selection part 727 in which the user selects and checks a score that the user agrees on when the user does not agree on the evaluation, and an assumed cause selection part 728 in which a cause assumed as a cause of a decrease in evaluation is selected in a case where the evaluation point is low, for example.

In the evaluation item display part 721, each evaluation item and a detailed evaluation item related thereto are displayed. Further, a point considered to be a problem is displayed. In the example illustrated in FIG. 7, the evaluation item display part 721 displays: an item of “preoperative (preparation) time” as an evaluation item; an item of “anesthesia intubation time” as a high evaluation item; and an item of “sensor mounting time on patient” as a low evaluation item. Similarly, items such as “intraoperative (manipulation) time”, “affected part deployment time”, and “suture time” are also displayed.

The evaluation item display part 721 may display items of the evaluation points calculated in the Formulas (1) to (13) in the order described above (the order of the display items is fixed), or may display the evaluation points in descending order or ascending order (the order of the display items is variable). Distinction between high evaluation and low evaluation may be made in the display such as display highlighted for items with low evaluation.

The moving image reproduction button 722 is a button operated at a time of reproducing a moving image corresponding to the item displayed in the evaluation item display part 721. For example, in a case where the moving image reproduction button 722 displayed on the right side of the item of the preoperative (preparation) time is operated, a moving image captured at a preparation time before surgery is reproduced.

The target case value display part 723 displays information about an evaluation case used in each evaluation. In the example illustrated in FIG. 7, for example, “58 mim” is displayed for the item of the preoperative (preparation) time, which indicates that a time corresponding to the preoperative (preparation) time of the evaluation target case is 58 minutes.

The information (time in FIG. 7) displayed in the target case value display part 723 may also function as a button, and a detailed graph or data may be expanded when a predetermined operation such as clicking is performed on the information as the button.

The reference value display part 724 displays a reference value used in each evaluation. For example, “56 mim” is displayed for the item of the preoperative (preparation) time, which indicates that a time to be the reference value corresponding to the preoperative (preparation) time of the evaluation target case is 56 minutes.

The evaluation result display part 725 displays an evaluation point. The evaluation point to be displayed may be the evaluation point itself calculated by Formulas (1) to (13) described above, but for example, an evaluation point obtained by conversion into five-stage evaluation may be displayed. FIG. 7 illustrates an example in which evaluation points when converted into five-stage evaluation are displayed. For example, a state is indicated in which the evaluation point of the item of the preoperative (preparation) time is “3”.

Note that, here, a case will be described as an example in which the evaluation point displayed in the evaluation result display part 725 is an evaluation point in the five-stage evaluation, but the number of stages of the stage evaluation is not limited to five stages, and may be ten stages, for example. Furthermore, a score converted into a score out of a maximum of 100 points may be displayed as the evaluation point. Furthermore, display such as “good”, “normal”, and “bad” may be used.

In a case where the evaluation points calculated by Formulas (1) to (13) are converted into the five-stage evaluation and displayed, for example, the following evaluation table is referred to for converting into the five-stage evaluation.

### Evaluation Table

5: Evaluation point ≤0.5
4: 0.5<Evaluation point ≤0.8
3: 0.8<Evaluation point ≤1.2
2: 1.2<Evaluation point ≤1.5
1: 1.5<Evaluation point

The evaluation table may be provided for each evaluation point calculated by Formulas (1) to (13) (an evaluation table is prepared for each item), or one or a plurality of evaluation tables may be shared.

The above-described evaluation table is, for example, a table for evaluation points of the sensor mounting time, and can be used in a case where the evaluation point is calculated with the following. Sensor mounting time of evaluation case/reference value

Note that a formula regarding the sensor mounting time is not shown in Formulas (1) to (13), but the sensor mounting time can also be set as an evaluation target by providing such a formula. Furthermore, the sensor mounting time is, for example, a time included in a preparation time of surgery before surgery, and is a time included in the preoperative (preparation) time.

Regarding the preoperative (preparation) time, it has been described that the evaluation point is calculated by Formula (1). However, when the preoperative (preparation) time is classified in more detail, as illustrated in FIG. 7, the anesthesia intubation time, the sensor mounting time on the patient, and the like are included, and it is also possible to calculate the evaluation point for each of the times and present the evaluation point to the user.

For example, in a case where the sensor mounting time of the evaluation case is 10 minutes and the reference value is 5 minutes, the evaluation point is calculated to be 2 (=10/2). When the evaluation table described above is referred to and conversion is performed into five-stage evaluation, 1 is obtained. This 1 is displayed in the evaluation result display part 725 of the screen as illustrated in FIG. 7.

The feedback input part 712 is configured to enable the user to determine whether or not a problem presented by the problem presentation part 711, specifically, the evaluation point displayed in the evaluation result display part 725 is appropriate, and reflect a determination result.

The agreement confirmation check part 726 of the feedback input part 712 is provided with a check box. The user checks the check box in a case where the user agrees on the evaluation point in the item, and the user selects and checks one of the scores 1, 2, 3, 4, and 5 displayed in the score selection part 727 in a case where the user does not agree on the evaluation point.

Note that, in a case where the check box of the agreement confirmation check part 726 is not checked and the check box of the score displayed in the score selection part 727 is not checked, it is processed that the user has agreed.

For example, the check box of the agreement confirmation check part 726 associated with the item of the preoperative (preparation) time is not checked because the user has not agreed, and the score “4” of the score selection part 727 is checked. That is, in this case, the user has not agreed on the evaluation point of “3” displayed in the evaluation result display part 725, has determined that “4” is valid, and has selected and checked the score of “4”. In this manner, a mechanism is provided in which the user determines validity of the evaluation point, and the user can input an evaluation point that can be determined to be valid in a case where the user determines that the evaluation point is invalid.

The assumed cause selection part 728 is provided that enables input of an assumed cause as a cause of an evaluation point calculated to be low, in a case where the user determines that the evaluation point is invalid. For example, in the example illustrated in FIG. 7, since the evaluation point associated with the item of the sensor mounting time on the patient is “1” and the user does not agree on the evaluation point, the check box of the agreement confirmation check part 726 is not checked. Furthermore, in the score selection part 727, the score of “5” is selected.

Since the button (in the screen example of FIG. 7, a triangular button displayed on the right side) displayed in the assumed cause selection part 728 of the item of the sensor mounting time on the patient is operated, a pull-down menu is displayed as illustrated in FIG. 7. The user can select, from items displayed in the pull-down menu, a cause that can be assumed as a cause of the evaluation point calculated to be low. Furthermore, at a lower part of the pull-down menu, a comment field is also provided in which a comment can be freely input in a case where the assumed cause is not in the pull-down menu.

In the example illustrated in FIG. 7, a comment “special equipment is used” is written as the assumed cause in the free comment field. As described above, a mechanism is provided in which, when the user does not agree on the evaluation point, more accurate evaluation is performed by allowing the user to input the cause.

Note that, displaying the pull-down menu when the button of the assumed cause selection part 728 is operated may be limited only to a case where the check box of the agreement confirmation check part 726 is not checked. The items displayed in the pull-down menu may be configured such that a different menu is displayed for each item displayed in the evaluation item display part 721, or may be configured such that the same menu is displayed.

In step S105 (FIG. 5), in a case where the feedback is received and it is determined that a button 730 of reevaluation is operated, the processing proceeds to step S106.

Note that, when a return button 729 is operated, the screen displayed on the display 502 is switched to the screen illustrated in FIG. 6, the processing returns to step S101, and the state is returned again to the state where a search condition related to searching for an evaluation case to be an evaluation target and a reference case as a comparison target is input.

In step S106, it is determined whether or not the search condition has been changed. It is determined that the search condition has been changed when there is an item selected in the pull-down menu of the assumed cause selection part 728 or when a comment is written in the free comment field. In a case where it is determined in step S106 that the search condition has been changed, the processing returns to step S102, and the subsequent processing is repeated.

The update unit 617 (FIG. 4) adds the item selected or the comment input in the assumed cause selection part 728 as a new search condition, and selects a reference case. For example, as illustrated in FIG. 7, in a case where a comment such as “special equipment is used” is written, the search is performed again after adding the search condition such as “special equipment is used” to the search condition input on the screen illustrated in FIG. 6.

For example, it is assumed that, in the first search, 20 cases are obtained by searching, evaluation points are calculated with 20 cases used as reference cases, and a problem point presentation screen as illustrated in FIG. 7 is presented to the user. Then, as described above, in a case where an assumed cause such as “special equipment is used” is input to the assumed cause selection part 728, a second search is performed after adding a new search condition such as “special equipment is used”. As a result, when five cases are obtained by searching, the evaluation point of the evaluation case set as the evaluation target is calculated again using the five cases set as reference cases.

For example, in a case where 1 is obtained in the first evaluation for the item of the sensor mounting time on the patient described above, and in a case where the reference value becomes 12 minutes in the reevaluation, the evaluation point is 0.83 (=10/12). When the evaluation point is converted into five-stage evaluation on the basis of the evaluation table, the evaluation point is updated to 3.

The screen including the problem presentation part 711 and the feedback input part 712 as illustrated in FIG. 7 is presented to the user by using the recalculated evaluation point. As described above, in a case where there is a special circumstance in the evaluation target case, such circumstance is input by the user involved in the evaluation target, and an evaluation point considering such circumstance is calculated.

In a case where it is determined in step S106 that the search condition has not been changed, the processing proceeds to step S107. In step S107, a total point is presented. The total point is final evaluation (score) of the evaluation target case calculated using the evaluation point displayed for each item.

Since the total point is calculated after the feedback as described above, the total point is a score reflecting the result of the feedback. That is, in a case where some cause is input to the assumed cause selection part 728, the total point is calculated after update to the evaluation point in consideration of the cause. Therefore, even in a case having a special circumstance, the total point can be obtained in consideration of the circumstance.

In a case where there is a score selected by the score selection part 727, in other words, in a case where there is an evaluation point on which the user does not agree, the total point may be calculated using the evaluation point (a score displayed in the evaluation result display part 725) on which the user has not agreed, or the total point may be calculated using the score selected by the user (the score checked in the score selection part 727).

A mechanism may be provided in which arbitrary feedback for improving the total point is not reflected in the total point. When the feedback is received or the feedback result is reflected, and reevaluation is performed, for example, there is a possibility that the feedback may be performed to rewrite to a good score in order to obtain a good result. Therefore, a mechanism for preventing such arbitrary feedback from being performed may be provided.

For example, a mechanism may be provided in which a user who is not involved in surgery cannot perform feedback, by limiting the evaluation case to only surgery related to the user. A configuration may be adopted in which, since the evaluation itself for the evaluation case can be changed, only the user involved in the surgery can make such a change, and the user who is not involved in the surgery, in other words, who does not know whether or not there is a special circumstance in the surgery is excluded.

In order to limit the user who can perform feedback, only surgery related to the user can be selected as the evaluation case. For example, when the information processing system 1 is used, login with a user ID or a password is performed, and the user is specified. Then, a mechanism may be provided in which only a case related to the specified user can be input into the evaluation case input field 701 (displayed as the pull-down menu) (FIG. 6).

The user who can perform feedback can be a user registered in advance, for example, a user in a controlling position such as a surgery unit manager or a nurse manager. Furthermore, the user who can perform feedback is a medical worker involved in the target case. Input authority to perform feedback can be given to these users in advance so that the user who can perform feedback can be limited.

Note that, in a case where such a mechanism capable of limiting the user who can perform feedback is provided, a mechanism may be provided in which a screen is provided on which evaluation of a predetermined case can be simply viewed although the feedback processing cannot be performed, and a user who is not involved in the surgery can also view the evaluation. By providing such a mechanism, it is possible to create a database of problem points and share information among medical workers.

A mechanism that can limit the user who can perform feedback may be provided, and calculation of a total point may be performed after a result of feedback given by a plurality of people is obtained. The total evaluation (total point) may be updated only when feedback given by a plurality of people is acquired so that the total evaluation does not change due to one person's feedback.

In a case where a result of feedback is extremely different from a result of other feedback, for example, in a case where a plurality of users gives feedback indicating that evaluation points of 1 to 3 are valid, and feedback such as “5” is given, such extremely different feedback result may be excluded.

The score for each item selected by the score selection part 727, an average value thereof, and the like may be used for adjustment of the evaluation table. The evaluation table also affects calculation of the evaluation point (conversion into five-stage evaluation). Therefore, a mechanism may be provided in which adjustment of the evaluation table can be performed only by an authorized user, for example, an administrator, or the score for each item selected by the score selection part 727, the average value thereof, and the like are used for adjustment when a certain number of pieces of feedback is collected.

The total point can be calculated, for example, on the basis of the following Formulas (14) and (15).

$$Total point = ( 1 / ( water balance + cost + preoperative time + intraoperative time + postoperative time + BMI + blood pressure + past history + stability degree in surroundings of bed + stability degree outside of surroundings of bed + busy degree + total workload + task processing time achievment degree - task order matching degree - patient care degree ) \times 1000 ) / index reference value \qquad ( 14 )$$

$$Index reference value = 1 / ( 1 + 1 + 1 + 1 + 1 + 1 + 1 + 1 + 1 + 1 + 1 + 1 + 1 - 1 - 1 ) \times 1000 \qquad ( 15 )$$

The total point is to be a value obtained by subtracting each evaluation point calculated by Formulas (12) and (13) from a value obtained by adding individual evaluation points calculated in Formulas (1) to (11) (including patient backgrounds such as a BMI, a blood pressure, and a past history, but prognosis calculated by Formula (6) is excluded), dividing 1 by the resulting value, multiplying the resulting value by 1000, and dividing the resulting value by the index reference value. In a case of calculating the evaluation point for 15 items, the index reference value is to be a value obtained by multiplying, by 1000, a value obtained by dividing 1 by 11.

In Formulas (14) and (15), the evaluation point for addition is an evaluation point of an item that is better as the evaluation point is smaller, and corresponds to the items calculated by Formulas (1) to (11). On the other hand, the evaluation point for subtraction is an evaluation point of an item that is better as the evaluation point is larger, and corresponds to the items calculated by Formulas (12) and (13).

In Formulas (14) and (15) for calculating the total point, the total point may be calculated by performing weighting. An example thereof is shown in the following Formulas (16) and (17).

$$Total point = ( 1 / water balance \times 1.3 + cost \times 1.5 + preoperative time \times 1 + intraoperative time \times 2 ) + postoperative time \times 1 + BMI \times 1.3 + blood pressure \times 1.3 + past history \times 1.3 + stability degree in surroundings of bed \times 1 + stability degree outside of surroundings of bed \times 1.2 + busy degree \times 1 + total workload \times 1 + task processing time achievment degree \times 1.1 - task order matching degree \times 1.3 - patient care degree \times 1.5 ) \times 1000 ) / index reference value \qquad ( 16 )$$

$$Index reference value = 1 / ( 1 \times 1.3 + 1 \times 1.5 + 1 \times 1 + 1 \times 2 + 1 \times 1 + 1 \times 1.3 + 1 \times 1.3 + 1 \times 1.3 + 1 \times 1 + 1 \times 1.2 + 1 \times 1 + 1 \times 1 + 1 \times 1 - 1 \times 1.3 - 1 \times 1.5 ) \times 1000 \qquad ( 17 )$$

The weighting value is an example, and the description does not indicate limitation. The total point calculated on the basis of Formula (14) or Formula (16) described above is presented to the user as a final score for the evaluation case.

The total point calculated on the basis of Formula (14) or Formula (16) described above may be presented to the user as it is, or may be converted into five-stage evaluation similarly to the evaluation point, and the score in the five-stage evaluation may be presented to the user. In a case where the score in the five-stage evaluation is presented to the user, the stage evaluation may be different from the evaluation point, such as ten-stage evaluation, instead of the five-stage evaluation. Furthermore, the total point may be converted into a score out of 100 points to be presented to the user.

The score for each item selected by the score selection part 727, an average value thereof, and the like may be used for adjustment of the weighting value. Since the weighting value affects calculation of the total point, a mechanism may be provided in which adjustment of the weighting value can be performed only by an authorized user, for example, an administrator, or the score for each item selected by the score selection part 727, the average value thereof, and the like are used for adjustment when a certain number of pieces of feedback are collected.

As described above, according to the present technology, evaluation of a predetermined case can be presented to a user. By viewing the presented evaluation, the user can recognize, for example, an item with low evaluation, and can consider and execute a means for improving as a result of the recognition. By repeating such processing, it is possible to further improve the efficiency of the surgery-related work.

According to the present technology, feedback from the user can be received for the presented evaluation, and the reevaluation after the feedback can be presented to the user. The accumulated evaluation for surgery-related cases can be updated, and evaluation suitable for various cases can be appropriately presented.

According to the present technology, it is easy to recognize best practices for each hospital, case, surgeon, task, index, and the like. Since feedback can be performed by answering a proposal or a question, it is easy to input feedback. The customization of the calculation of the index such as the evaluation point and the total point (customization of the evaluation table and the weighting value) can be performed without bothering the user, and individual adjustment can be made unnecessary.

The display of the problem point described above can be used not only as a reference for improving the efficiency of the operating room but also for reflective learning of each medical worker. It is possible to create a database of problem points and share information.

### <Recording Medium>

The above-described series of processes can be performed by hardware or software. In a case where the series of processing is executed by software, a program constituting the software is installed in a computer. Here, examples of the computer include a computer incorporated in dedicated hardware, and for example, a general-purpose personal computer that can execute various functions by installing various programs.

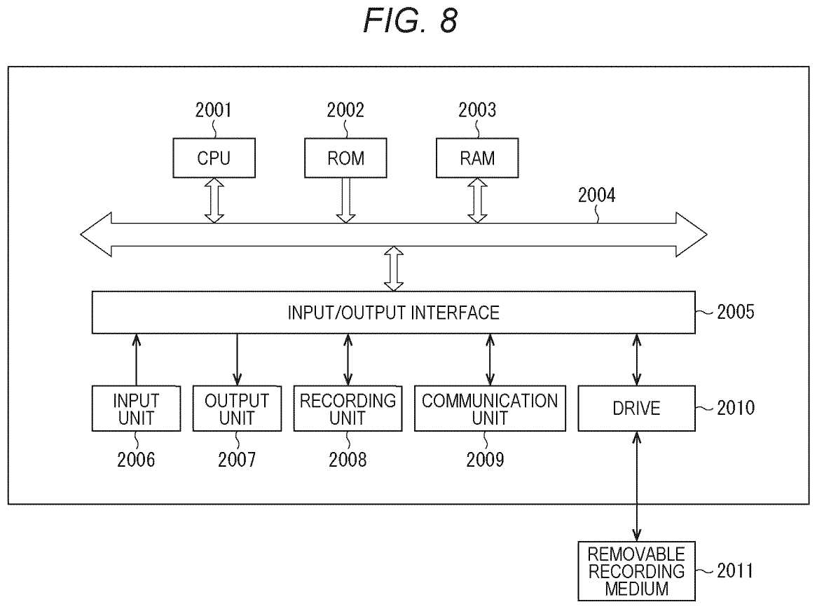

FIG. 8 is a block diagram illustrating a configuration example of hardware of a computer that executes the above-described series of processing with a program. In the computer, a central processing unit (CPU) 2001, a read only memory (ROM) 2002, and a random access memory (RAM) 2003 are connected to one another by a bus 2004. An input/output interface 2005 is further connected to the bus 2004. An input unit 2006, an output unit 2007, a storage unit 2008, a communication unit 2009, and a drive 2010 are connected to the input/output interface 2005.

The input unit 2006 includes a keyboard, a mouse, a microphone, and the like. The output unit 2007 includes a display, a speaker, and the like. The storage unit 2008 includes a hard disk, a nonvolatile memory, and the like. The communication unit 2009 includes a network interface and the like. The drive 2010 drives a removable medium 2011 such as a magnetic disk, an optical disk, a magneto-optical disk, or a semiconductor memory.

In the computer designed as described above, the CPU 2001 loads, for example, a program stored in the storage unit 2008 into the RAM 2003 via the input/output interface 2005 and the bus 2004, and executes the program, so that the series of processes described above is performed.

The program executed by the computer (CPU 2001) can be provided by being recorded in the removable medium 2011 as a package medium or the like, for example. Furthermore, the program can be provided via a wired or wireless transmission medium such as a local area network, the Internet, or digital satellite broadcasting.

In the computer, the program can be installed in the storage unit 2008 via the input/output interface 2005 by mounting the removable medium 2011 on the drive 2010. Furthermore, the program can be received by the communication unit 2009 via a wired or wireless transmission medium and installed in the storage unit 2008. In addition to this, the program can be installed in the ROM 2002 or the storage unit 2008 in advance.

Note that the program executed by the computer may be a program in which processing is performed in time series in the order described in the present specification or may be a program in which processing is performed in parallel or at necessary timing such as when a call is made.

In the present specification, the system represents the entire device including a plurality of devices.

Note that the effects described in the present description are merely examples and are not limited, and other effects may be provided.

Note that the embodiments of the present technology are not limited to the above-described embodiments, and various changes can be made without departing from the gist of the present technology.

Note that the present technology can also have the following configurations.

(1)

An information processing device including:
an analysis unit configured to analyze data regarding surgery;
a search condition acquisition unit configured to acquire a search condition for searching for a reference case to be compared with an evaluation case as an evaluation target;
a search unit configured to search for the reference case matching the search condition from a database in which results of analysis by the analysis unit are accumulated;
a calculation means configured to calculate an evaluation point of the evaluation case by using the evaluation case and the reference case, the reference case being obtained by searching with the search unit;
a presentation means configured to present the evaluation point calculated by the calculation means to a user;
a feedback acquisition means configured to acquire feedback given by the user on the evaluation point presented by the presentation means; and
an update unit configured to update the evaluation point obtained by the calculation means, in a case where feedback is acquired by the feedback acquisition means.
   (2)

The information processing device according to (1) above, in which the feedback acquisition means acquires a cause of the evaluation point calculated to be low, and the update unit searches the database again after adding the cause to the search condition, and the calculation means recalculates an evaluation point of the evaluation case by using the reference case obtained by searching again.

(3)

The information processing device according to (2) above, in which
the feedback acquisition means displays a list of items assumed as the cause, and acquires an item selected by the user from the listed items.
   (4)

The information processing device according to (2) above, in which
the feedback acquisition means acquires a comment input by the user as the cause.
   (5)

The information processing device according to any one of (1) to (4) above, in which
in a case where the user does not agree on the evaluation point, the feedback acquisition means acquires a score input by the user as a score on which the user agrees.
   (6)

The information processing device according to any one of (1) to (5) above, in which
the calculation means calculates the evaluation point of the evaluation case on the basis of a predetermined formula using an average value of the reference case.
   (7)

The information processing device according to any one of (1) to (6) above, further including:
a total point calculation unit configured to calculate a total point for the evaluation case by using the evaluation point calculated by the calculation means for each of a plurality of items.
   (8)

The information processing device according to (7) above, in which
the total point calculation unit calculates the total point by using the evaluation point updated by the update unit.
   (9)

The information processing device according to (7) above, in which
the total point calculation unit calculates the total point after the feedback is acquired from a plurality of users.
   (10)

The information processing device according to any one of (1) to (9) above, in which
the calculation means converts the evaluation point into a predetermined stage evaluation, and the presentation means presents the evaluation point converted into the predetermined stage evaluation.
   (11)

The information processing device according to any one of (1) to (10) above, in which
the feedback acquisition means acquires feedback from a user involved in the evaluation case.
   (12)

An information processing method including,
by an information processing device:
analyzing data regarding surgery;
acquiring a search condition for searching for a reference case to be compared with an evaluation case as an evaluation target;
searching for the reference case matching the search condition from a database in which results of analysis are accumulated;
calculating an evaluation point of the evaluation case by using the evaluation case and the reference case, the reference case being obtained by the searching;
presenting the calculated evaluation point to a user;
acquiring feedback given by the user on the presented evaluation point; and
updating the evaluation point in a case where the feedback is acquired.
   (13)

A program for causing a computer to execute processing including a step of:
analyzing data regarding surgery;
acquiring a search condition for searching for a reference case to be compared with an evaluation case as an evaluation target;
searching for the reference case matching the search condition from a database in which results of analysis are accumulated;
calculating an evaluation point of the evaluation case by using the evaluation case and the reference case, the reference case being obtained by the searching;
presenting the calculated evaluation point to a user;
acquiring feedback given by the user on the presented evaluation point; and
updating the evaluation point in a case where the feedback is acquired.

### REFERENCE SIGNS LIST

1 Information processing system
100 In-hospital system
101 OR system
200 Data source
201 Sensor
202 In-OR device
203 Operative field video acquisition unit
300 Server
301 Database
302 Surgery progression simulator
311 Resource information
312 Information
400 Processing device
401 Data processing unit
402 Situation determination unit
500 Information presentation system
501 Information presentation processing unit
502 Display
611 Data analysis unit
612 Case search condition acquisition unit
613 Case search unit
614 Evaluation point calculation unit
615 Problem point presentation unit
616 Feedback acquisition unit
617 Update unit
618 Total point presentation unit
701 Evaluation case input field
702 Reference case input field
711 Problem presentation part
712 Feedback input part
721 Evaluation item display part
722 Moving image reproduction button
723 Target case value display part
724 Reference value display part
725 Evaluation result display part
726 Agreement confirmation check part
727 Score selection part
728 Assumed cause selection part
729 Button
730 Button

1. An information processing device comprising:
an analysis unit configured to analyze data regarding surgery;
a search condition acquisition unit configured to acquire a search condition for searching for a reference case to be compared with an evaluation case as an evaluation target;
a search unit configured to search for the reference case matching the search condition from a database in which results of analysis by the analysis unit are accumulated;
a calculation means configured to calculate an evaluation point of the evaluation case by using the evaluation case and the reference case, the reference case being obtained by searching with the search unit;
a presentation means configured to present the evaluation point calculated by the calculation means to a user;
a feedback acquisition means configured to acquire feedback given by the user on the evaluation point presented by the presentation means; and
an update unit configured to update the evaluation point obtained by the calculation means, in a case where feedback is acquired by the feedback acquisition means.

2. The information processing device according to claim 1, wherein
the feedback acquisition means acquires a cause of the evaluation point calculated to be low, and
the update unit searches the database again after adding the cause to the search condition, and
the calculation means recalculates an evaluation point of the evaluation case by using the reference case obtained by searching again.

3. The information processing device according to claim 2, wherein
the feedback acquisition means displays a list of items assumed as the cause, and acquires an item selected by the user from the listed items.

4. The information processing device according to claim 2, wherein
the feedback acquisition means acquires a comment input by the user as the cause.

5. The information processing device according to claim 1, wherein
in a case where the user does not agree on the evaluation point, the feedback acquisition means acquires a score input by the user as a score on which the user agrees.

6. The information processing device according to claim 1, wherein
the calculation means calculates the evaluation point of the evaluation case on a basis of a predetermined formula using an average value of the reference case.

7. The information processing device according to claim 1, further comprising:
a total point calculation unit configured to calculate a total point for the evaluation case by using the evaluation point calculated by the calculation means for each of a plurality of items.

8. The information processing device according to claim 7, wherein
the total point calculation unit calculates the total point by using the evaluation point updated by the update unit.

9. The information processing device according to claim 7, wherein
the total point calculation unit calculates the total point after the feedback is acquired from a plurality of users.

10. The information processing device according to claim 1, wherein
the calculation means converts the evaluation point into a predetermined stage evaluation, and
the presentation means presents the evaluation point converted into the predetermined stage evaluation.

11. The information processing device according to claim 1, wherein
the feedback acquisition means acquires feedback from a user involved in the evaluation case.

12. An information processing method comprising,
by an information processing device:
analyzing data regarding surgery;
acquiring a search condition for searching for a reference case to be compared with an evaluation case as an evaluation target;
searching for the reference case matching the search condition from a database in which results of analysis are accumulated;
calculating an evaluation point of the evaluation case by using the evaluation case and the reference case, the reference case being obtained by the searching;
presenting the calculated evaluation point to a user;
acquiring feedback given by the user on the presented evaluation point; and
updating the evaluation point in a case where the feedback is acquired.

13. A program for causing a computer to execute processing comprising a step of:
analyzing data regarding surgery;
acquiring a search condition for searching for a reference case to be compared with an evaluation case as an evaluation target;
searching for the reference case matching the search condition from a database in which results of analysis are accumulated;
calculating an evaluation point of the evaluation case by using the evaluation case and the reference case, the reference case being obtained by the searching;
presenting the calculated evaluation point to a user;
acquiring feedback given by the user on the presented evaluation point; and
updating the evaluation point in a case where the feedback is acquired.
